(19) United States
(12) Patent Application Publication
(10) Pub. No.: US 2017/0246234 A1
(43) Pub. Date: Aug. 31, 2017

(54) METHODS OF USING WATER SOLUBLE TEA EXTRACTS TO IMPROVE MUSCLE PERFORMANCE, STRESS RESPONSE, AND RECOVERY FOLLOWING EXERCISE

(71) Applicant: Kemin Industries, Inc., Des Moines, IA (US)

(72) Inventors: Kelli Herrlinger, Johnston, IA (US); Kristin Nieman, Mitchelville, IA (US)

(21) Appl. No.: 15/445,034

(22) Filed: Feb. 28, 2017

Related U.S. Application Data
(60) Provisional application No. 62/301,420, filed on Feb. 29, 2016.

(51) Int. Cl. A61K 36/82

(52) U.S. Cl. CPC A61K 36/82

(57) ABSTRACT

The present invention relates to methods for improving muscle performance wherein a subject is administered an efficacious dose of a mixture of water soluble extracts of green and black tea. The invention also relates to a method of increasing antioxidant capacity and glutathione reductase while preventing the increased cortisol response in a subject and a method for improving testosterone levels in a subject following exercise, comprising the step of administering to a subject an efficacious dose of a mixture of water soluble extracts of green and black tea. Other aspects of the present invention relate to improving cortisol stress response and/or testosterone levels following exercise.

This application claims priority to U.S. Patent Application 62/301,420, filed Feb. 29, 2016, which is incorporated herein in its entirety by this reference.

### BACKGROUND OF THE INVENTION

The present invention relates generally to the effect of water soluble tea extract supplementation on muscles and, more specifically to the effect of water soluble tea extract supplementation on biomarkers of muscle damage, stress, performance and recovery of functional performance from acute high intensity resistance exercise.

Previous research has demonstrated an increase in free radical production following resistance exercise (Hudson et al., 2008). This has been demonstrated to have a profound effect on many bodily processes, including inflammation (Vassilakopoulos, Roussos, & Zakynthinos, 2005). Previous studies have examined the impact of oxidative stress on cytokine production, demonstrating conflicting results (Fischer et al., 2004; Petersen et al., 2001; Vassilakopoulos et al., 2003). It has been suggested that following concentric exercises (like cycling), antioxidant supplementation may result in reduced oxidative stress, which in turn may result in a reduced cytokine response (Vassilakopoulos, et al., 2003; Vassilakopoulos, et al., 2005). However, it is important to note that many of the other studies that examined this question utilizing concentric/eccentric protocols have used aerobic-based exercises as opposed to dynamic resistance exercise (Nieman et al., 2002; Petersen, et al., 2001).

It is generally understood that resistance exercise performed at a sufficient intensity will result in microtrauma to skeletal muscle, which may be reflected by leakage of various biomarkers (e.g., creatine kinase (CK) and/or myoglobin), increases in muscle soreness and potential decreases in muscle performance (Clarkson & Hubal, 2002; Paulsen et al., 2005). The mechanical stress associated with a resistance exercise stimulus and the resulting tissue damage signals a profound non-specific immune response (Freidenreich & Volek, 2012; Tidball & Villalta, 2010). This response manifests itself through increases in cytokine and chemokine production from skeletal muscle tissue, endothelial cells, resident macrophages, and other circulating immune cells (Della Gatta, Cameron-Smith, & Peake, 2014; Nieman et al., 2004). Once released, cytokines and chemokines will elicit a response from the immune system, resulting in an accumulation of myeloid cells within a few hours, which persist for several days (Paulsen et al., 2010).

The infiltration of damaged tissue consists of three phases; preliminary, early and late, with each phase eliciting specific actions within the recovery process (Tidball & Villalta, 2010). The preliminary phase, which promotes an inflammatory environment (Nguyen & Tidball, 2003; Pizza, Peterson, Baas, & Koh, 2005), primarily consists of neutrophils, which are the most abundant granulocyte (Parkin & Cohen, 2001). Granulocytes, which include neutrophils, eosinophils and basophils are produced within the bone marrow as a result of stimulation by granulocyte colony stimulating factor (G-CSF) (Roberts, 2005), while granulocyte-macrophage colony stimulating factor (GM-CSF) and interleukin-8 (IL-8) function to activate and recruit granulocytes to the site of tissue damage (Francisco-Cruz et al., 2014; Hammond et al., 1995). Following the preliminary phase, the early and late phases are characterized by macrophages that promote inflammation (M1), and recovery (M2), respectively (Tidball & Villalta, 2010).

The inventors were aware of only one study that has examined antioxidant capacity and the inflammation response using a resistance exercise protocol (leg extensions), which demonstrated decreased cytokine concentrations following high volume leg extensor exercise (Fischer, et al., 2004). Therefore, the examination of antioxidants and how they interact with the immune system following a dynamic resistance exercise protocol, has not previously been explored, specifically the efficacy of antioxidant supplementation while limiting the effect aerobic activity has on oxidative stress (Nieman, et al., 2002; Petersen, et al., 2001).

While resistance exercise is known to increase oxidative stress (Hoffman et al., 2007; Ramel, Wagner, & Elmadfa, 2004), it has also been demonstrated to increase antioxidant capacity immediately following exercise by increasing lipid soluble antioxidants (Ramel, et al., 2004). This has been demonstrated for both high volume and high intensity resistance exercise protocols (Hoffman, et al., 2007; Hudson, et al., 2008). Limited data have been presented demonstrating a direct benefit of increased antioxidant capacity on exercise performance; however, free radical production in the form of reactive oxygen species has been demonstrated to reduce exercise performance and tolerance (Reid & Durham, 2002; Watson et al., 2005). Additionally, the acute increase in antioxidant capacity following resistance exercise may differ based on resistance training status; untrained individuals obtain greater antioxidant capacity following acute resistance exercise, relative to trained individuals (Ramel, et al., 2004). Therefore, antioxidant supplementation employed to counteract the natural rise in free radicals and reduce the oxidative response associated with resistance exercise (Hoffman, et al., 2007; Hudson, et al., 2008), may be more beneficial in highly trained individuals. It is possible that resistance exercise, in combination with a water extracted tea extract, may improve adaptation by reducing the relative oxidative stress after exercise. Thus the purpose of this study is to evaluate the efficacy of an antioxidant supplement, a water extracted tea extract (XSurge [XS]), on stress, performance and acute responses to resistance exercise and how these responses are modified following resistance training.

### SUMMARY OF THE INVENTION

The study design was a randomized, double-blind, placebo-controlled, with an additional control group that is not blinded. The study was conducted following the International Conference on Harmonisation (ICH) Guidelines and in accordance with the Declaration of Helsinki. Institutional Review Board (IRB) approval (New England IRB) was acquired prior to conducting any protocol-specific procedures.

Healthy, recreationally active (<3 h of structure exercise/week) male subjects, 18-35 years (N=40) of age, with body mass index 18.0-34.9 kg/m², inclusive, were supplemented with one of the following treatments: (a) Active: XSurge (XS, proprietary blend of water-extracted black and green tea extracts), 2 capsules b.i.d, with food (total: 2 g/day, 4 capsules/day); (b) Placebo (PL, n=15), micro cellulose capsules matched for capsule size and color, 2 capsules b.i.d, with food (total: 2 g/day, 4 capsules/day); or (c) control (CON, n=10; Phase I only): no treatment.

Phase I consisted of baseline (BL) testing, 28 days of daily supplementation with 2 g (500 mg capsules; 4 capsules/day) XS, and an acute damaging protocol. The acute damaging protocol (AP1) consisted of five visits, of which, the first visit (D1) consisted of maximal strength testing. The second visit (D2) was at least 72 hours after D1, and consisted of function testing, blood draws and muscle biopsies, along with a muscle damaging resistance exercise bout. D3, D4 and D5 were completed 24, 48 and 96 hours later, and consisted of functional testing and blood draws, with a muscle biopsy at D4. Outcome Measures: BL measures consisted of functional testing and assessment of circulating measures. AP1 measures consisted of function testing, muscle tissue sampling, circulating markers as well as white blood cell surface expression of specific antigens.

Phase II consisted of six weeks of supervised resistance training (full body, three days per week with at least one day between each session) completed in the Human Performance Laboratory (HPL) at the University of Central Florida (UCF). Following the six weeks of training, subjects completed a second acute damaging protocol (AP2), which was conducted in the same fashion as the first acute damaging protocol. The second acute protocol did not have any muscle biopsies. Outcome Measures: AP2 measures consisting of function testing, circulating markers, as well as white blood cell surface expression of specific antigens.

### BRIEF DESCRIPTION OF THE DRAWINGS

FIG. 1 is a schematic representation of the study overview.

FIGS. 2A and 2B depict the gating procedures for CD14+ cells. FIG. 2A depicts CD14+ cells selected from the specified region based on forward (FSC) and side scatter (SSC) properties. FIG. 2B depicts the two dimensional histogram displaying fluorescence characteristics of cells in selected region. Cells positive for CR3 and CD14 are displayed in the upper right quadrant.

FIG. 3 is a schematic representation of the acute damaging protocol overview.

FIG. 4 is a chart of the Isokinetic Leg Extension EMG-RMS Measurement at slow (30° /sec) speed. Mean±SEM, ANOVA Overall Model Interaction P=0.003, *PL vs. XS (pre-exercise value in AP1-referred to here at post supplementation) P=0.006. Intent to Treat Group.

FIG. 5 is a chart of the Isokinetic Leg Extension EMG-RMS Measurement at slow (180°/sec) speed. Mean ±SEM, ANOVA Overall Model Interaction P=0.017, ̂PL vs. XS (pre-exercise value in AP1-referred to here at post supplementation) P=0.020. Intent to Treat Group.

FIG. 6 is a chart of the effects of supplementation on antioxidant capacity. Mean±SEM: ANOVA Overall Model Interaction P=0.010, *Change from Baseline P=0.007. Intent to Treat Group.

FIG. 7 is a chart of the effects of supplementation on cortisol. Mean±SEM: ANOVA Overall Model Interaction P=0.007, *Change from Baseline P=0.027. Intent to Treat Group.

FIG. 8 is a chart of the effect of supplementation on peak torque at 96 hours post acute damaging protocol. Mean±SEM, ANOVA Overall Model Interaction P=0.003, *CON vs. PL P=0.023. CON vs. XS NS. XS peak power no different than control group that did not do the acute muscle damaging protocol. Intent to Treat Group.

FIG. 9 is a chart of the recovery at 1 hour from an acute muscle damaging protocol as measured by Vertical Jump on a Force Plate. Mean±SEM: ANOVA Overall Model Interaction P=0.035, *CON vs. PL (1Hr) P=0.004. CON vs. XS NS. Overall between group comparison significant at 1Hr, P=0.013. XS peak power no different than the control group that did not do the acute muscle damaging protocol. Intent to Treat Group.

FIG. 10 is a chart of the effect of 28 days of supplementation on Testosterone following an acute muscle damaging protocol. Mean±SEM: ANOVA Overall Model Interaction P=0.047, Between Group Comparison immediately post exercise (IP) P=0.019, *CON vs. XS (IP) P=0.006. CON vs. PL NS, XS vs. PL P=0.094. Intent to Treat Group.

FIG. 11A is an intramuscular protein content of granulocyte colony stimulating factor (G-CSF). FIG. 11B is an intramuscular protein content of granulocyte-macrophage colony stimulating factor (GM-CSF), and (C.) Interleukin-8 (IL-8). XSurge (XS), Placebo (PL) and Control (CON) were analyzed for intramuscular protein content prior to exercise (PRE), as well as 1-(1H), 5-(5H) and 48-(48H) hours post exercise. * Significantly different than PRE (p<0.05); ̂ Significantly different than XS (p<0.05). Data presented as Mean±SD.

FIG. 12A depicts circulating granulocyte proportions and FIG. 12B depicts the adhesion characteristics. XSurge (XS), placebo (PL) and control (CON) groups were analyzed for changes in the granulocyte characteristics pre exercise (PRE), as well as immediately (IP), one-(1H), five-(5H), 24-(24H), and 48-(48H) hours post exercise. * Significantly different than corresponding value for PRE (p<0.05); A Significantly different than corresponding value for CON (p<0.05); # Significantly different than corresponding value for PL (p<0.05). Data presented as Mean±SD.

### DESCRIPTION OF THE INVENTION

Abbreviations

| Abbreviation | Definition |
|---|---|
| 1-RM | One-Repetition Maximum |
| ACSM | American College of Sports Medicine |
| AE | Adverse Event |
| AED | Automated External Defibrulator |
| ANOVA | Analysis of Variance |
| AP1 | Acute Damaging Protocol 1 |
| AP2 | Acute Damaging Protocol 2 |
| b.i.d. | Taken twice daily |
| BL | Baseline |
| CK | Creatine Kinase |
| CPR | Cardiopulmonary Ressucitation |
| CRF | Case Report Form |
| CSCS | Certified Strength and Conditioning Specialist |
| D1 | Acute Damaging Protocol Day 1 |
| D2 | Acute Damaging Protocol Day 2 |
| D3 | Acute Damaging Protocol Day 3 |
| D4 | Acute Damaging Protocol Day 4 |
| D5 | Acute Damaging Protocol Day 5 |
| DNA/RNA | Deoxy-Ribonucleic Acid |
| DNP | Dinitrophenylhydrazine |
| ELISA | Enzyme Linked Immunosorbent Assay |
| EMD | Electromechanical Delay |
| EMG | Electromyography |
| FRAP | Ferric Reducing Ability of Plasma |
| GSH | Glutathione |
| HIPPA | Health Insurance Portability and Accountability Act of 1996 |
| HPL | Human Performance Laboratory |
| ICH | International Conference on Harmonisation |
| IRB | International Review Board |
| MEDPF | Median Power Frequency |
| MITT | Modified Intent to Treat |
| MPF | Mean Power Frequency |
| MVIC | Maximal Voluntary Isometric Contraction |
| NME | Neuromuscular Economy |
| NSCA | National Strenght and Conditioning Association |
| PAR-Q | Physical Activity Readiness Questionnaire |
| PerP | Per Protocol |
| PI | Peak Impulse |
| PKF | Peak Force |
| PKT | Peak Torque |
| PL | Placebo |
| PP | Peak Power |
| RFD | Rate of Force Development |
| RMS | Root Mean Square |
| RPD | Rate of Power Development |
| SAE | Significant Adverse Event |
| SOD | Superoxide Dismutase |
| SPSS | Statistical Package for Social Sciences |
| TBARS | Thiobarbituric Acid Reactive Substances |
| XS | X-Surge |

Study Design

### Phase I Overview

Subjects reported to the HPL for baseline (BL) testing (See section 5.4.3). Afterwards, subjects began a 4-week supplementation protocol (See section 5.4.4). After supplementation (2 g/day), subjects completed their first acute damaging protocol (AP1) (described below).

Phase II Overview

At the conclusion of phase I, participants completed the training protocol (phase II), if they were not enrolled in the CON group, and continued supplementation at 2 g per day for the remaining 6 weeks. The training protocol (described below) consisted of resistance training 3 days per week for the duration of 6 weeks, except for the last week, which only consisted of 2 training days. At the conclusion of the 6 weeks the acute damaging protocol was repeated (AP2) with the exclusion that muscle biopsies were not conducted. Furthermore, one serving (1 g; 2 capsules) of XS was supplemented 1 hr prior to every training session with the snack.

Baseline (BL) Testing

Baseline (BL) testing consisted of a resting blood draw, as well as all performance measures, and anthropometric measures (height, weight, skinfolds). After BL testing was completed, participants were given their supplement (XS or PL). This timepoint commenced Phase I. For greater detail regarding these protocols, please see section 5.11. Participants were instructed to abstain from exercise and alcohol consumption for 72 hours prior to BL, as well as to abstain from food/caffeine intake for 12 hours prior to BL. Additionally, subjects were asked to ensure at least 8 hours of sleep the night before BL.

Supplementation

Subjects were supplemented with XS or PL daily. Participants were asked to report to the HPL five days per week for their supplement. Participants took one dose of the supplement in the lab, and were given their specified supplement in individual containers for the days they do not report to the lab. Participants were asked to return the empty containers upon their next visit to the lab.

Acute Damage Protocols (Phase I and II)

At the end of Phase I and II, participants in the XS and PL groups completed a lower body resistance exercise session on D2. Participants were instructed to abstain from exercise and alcohol consumption for 72 hours prior to D2, as well as to abstain from food/caffeine intake for 12 hours prior to D2. Additionally, subjects were asked to obtain at least 8 hours of sleep the night before D2-D5. After the blood sample was obtained, participants were provided the snack and one dose (1000 mg; 2 capsules) of the study supplement. After consumption, the remaining resting measures were obtained (Functional testing, and biopsy; described in section 5.11), and participants completed a general and specific warm up. The general warm up consisted of riding a cycle ergometer for 5 minutes at his preferred resistance. The specific warm up consisted of 10 body weight squats, 10 alternating lunges, 10 walking knee hugs and 10 walking butt kicks. The resistance exercise session included the squat, leg press and leg extension exercises. Participants completed 6 sets of the squat exercise and 4 sets of the leg press and leg extension exercises. The load for each exercise was 70% of each participant's previously determined 1RM (which will be determined at D1). Each set required participants to complete 10 repetitions. The rest interval between each set was 90 seconds. Functional tests were completed at 1, 5, 24, 48 and 96 hours following completion of the acute damaging bout. Additionally, blood samples were obtained immediately after, as well as 1, 5, 24, 48 and 96 hours after the acute damaging bout, while biopsies were obtained at 1, 5 and 48 hours. Additionally, as subjects arrived to the lab in a fasted state, they were provided with a small snack prior to the workout, and a small meal between the 1H and 5H timepoints. Following Phase I (immediately prior to Phase II), the acute protocol included muscle biopsies, however, the acute protocol following Phase II did not. During D3-D5 subjects were provided the study supplement after completion of function testing, blood and biopsy sampling. After consumption of the study product, participants were provided the second dose of the study supplement for consumption later in the day. Participants were asked to return the empty package of the study supplement the following day.

Training Protocol (Phase II)

Participants in the XS and PL groups reported to the Strength and Conditioning Lab to complete 6 weeks of resistance training during Phase II of the study. Training took place 3 days/week with at least one day of rest in between exercise sessions. If a participant missed an exercise session, a make up session was scheduled on the weekend with lab staff to ensure that 17 total sessions were completed during the 6 week period, while still maintaining appropriate rest periods between sessions. One hour prior to each session, participants reported to the HPL for supplement consumption. One hour after consumption, participants completed a general and specific warm up. The general warm up consisted of riding a cycle ergometer for 5 minutes at the subjects' preferred resistance. The specific warm up consisted of 10 body weight squats, 10 alternating lunges, 10 walking knee hugs and 10 walking butt kicks. The training was a full body progressive resistance training protocol focusing on all major muscle groups of the body. The assigned load for each core exercise (Smith machine squat and bench press) was 80% of the previously determined 1RM. For the remaining assistance exercises, participants adjusted the load to achieve an 8-10 RM (approximately 80% of their maximal strength per exercise). Each training session was monitored by a Certified Strength and Conditioning Specialist (CSCS). All repetitions and loads were charted in a log book. The CSCS adjusted the training load for next training session based upon the participants performance. Upon successful completion of all required repetitions per exercise (i.e., 3 sets of 10), the training load was increased, 5-10 lbs for upper body exercises and 10-20 lbs for lower body exercises. The training protocol was designed according to the recommendations of the National Strength and Conditioning Association (NSCA). The 6-week whole body resistance training program presented here was repeated every week:

TABLE 1
Training Regimen for Phase II.

| Training 1 | | Training 2 | | Training 3 | |
|---|---|---|---|---|---|
| Smith Machine | 4 × 8-10 | Leg Press | 4 × 8-10 | Smith Machine | 4 × 8-10 |
| Squat | RM | | RM | Squat | RM |
| Bench Press | 4 × 8-10 | Incline bench | 4 × 8-10 | Bench Press | 4 × 8-10 |
| | RM | press | RM | | RM |
| Seated dumbbell | 3 × 8-10 | Dumbbell Step | 3 × 8-10 | Seated dumbbell | 3 × 8-10 |
| press | RM | ups | RM | press | RM |
| Low Row | 3 × 8-10 | Upright Rows | 3 × 8-10 | Low Row | 3 × 8-10 |
| | RM | | RM | | RM |
| Leg Extension | 3 × 8-10 | Lat Pulldown | 3 × 8-10 | Leg Extension | 3 × 8-10 |
| | RM | | RM | | RM |
| Leg Curl | 3 × 8-10 | Seated Calf | 3 × 8-10 | Leg Curl | 3 × 8-10 |
| | RM | Raise | RM | | RM |

Study Overview:

Patient Populations

Control Population: The control group (CON) was included in Phase I to quantify or describe the muscle damage that occurs with the damaging exercise bout. The CON group did not complete the resistance training protocol during Phase II and did not receive any supplement during Phases I and II. Further, the CON group did undergo biopsies and blood draws during Phase I. Given that several biological markers show diurnal patterns, a CON group that did not exercise or receive supplement would discern the damage that the exercise bout caused from diurnal fluctuations. In addition, the stress of biological sample collection can influence some of the markers. If this is the case, the CON group can help to identify this potential contribution to variation.

Supplemented Population: The experimental population included all subjects that were enrolled into the study in either the PL or XS groups. The Experimental Population was broken down into:

Modified Intent to Treat (MITT): The MITT population comprised all subjects who consumed at least one dose of study product, and provided at least one on-treatment outcome data point during Phase I or II.

Per Protocol (PerP): In addition, PerP population was comprised of a subset of the MITT population that completed all testing. Subjects were excluded from the PerP population for the following reasons:
Violations of inclusion or exclusion criteria that could influence the evaluation of response
Non-compliance by the subject, including, but not limited to:
Missing training sessions (80% completion or 14 of 17 sessions)
Less than 80% compliance with study product consumption
Failure to complete BL, AP1 and AP2 testing
Inclusion/Exclusion Criteria
Inclusion Criteria
Recreationally active. Non-resistance trained (participated in <1 weight training workout per week over the previous year) and participates in <3 h of total structured exercise/week as determined by the health and activity questionnaire.
Subject is judged by the Investigator to be healthy and free of any physical limitations (determined by health and activity questionnaire and PAR-Q)
Subject is male 18-35 years of age, inclusive
Subject has a body mass index of 18.0-34.9 kg/m², inclusive
Subject is willing to maintain habitual diet (including alcohol and caffeine consumption) throughout the study period
Subject is willing to abstain from dietary supplementation throughout the duration of the study
Subject is willing and able to engage in only the supervised moderate-intensity exercise throughout Phase II of the trial (3 sessions/week for 6 weeks)
Subject understands the study procedures and signs forms providing informed consent to participate in the study and authorization for release of relevant protected health information to the study Investigators

Exclusion Criteria
Subject is currently or will be enrolled in another clinical trial
Subject is a habitual consumer of tea defined as >8 oz/day of either green or black tea within the 14 days prior to the screening visit
Subject has a history or presence of a clinically relevant cardiac, renal, hepatic, endocrine (including diabetes mellitus), pulmonary, biliary, gastrointestinal, pancreatic, or neurologic disorder
Subject has a history or presence of cancer in the prior 2 years, except for non-melanoma skin cancer
Subject is unable to perform physical exercise (determined by health and activity questionnaire)
Subject is a current smoker or has quit within the last 6 months
Subject is engaged in an extreme diet including but not limited to, Atkins, South Beach, Intermittent Fasting, etc.
Subject is allergic to the study product or PL
Subject is taking any other nutritional supplement or performance enhancing drug (determined from health and activity questionnaire)
Subjects that have donated blood or plasma within the previous week
Subject has any chronic illness that causes continuous medical care
Taking any type of prescription or over-the-counter medication including but not limited to corticosteroids, non-steroidal anti-inflammatory drugs, and antibiotics within the 14 days prior to the screening visit.

Outcome Measure Assessments
Primary Outcome Variables:
   Peak Torque (as measured isokinetically at moderate speed by Biodex)
   Circulating Creatine Kinase (CK) concentration
   Circulating Cortisol concentration
Secondary Outcome Variables:
   Performance Measures
      Strength Testing
         Squat
         Leg Press
         Leg Extension
      Electromyography (EMG)
         Root Mean Square (Isokinetic)
         Electromechanical Delay (Isokinetic)
         Mean Power Frequency (Isokinetic)
         Median Power Frequency (Isokinetic)
         Root Mean Square (Isokinetic; 3 speeds)
         Mean Power Frequency (Isokinetic; 3 speeds)
         Median Power Frequency (Isokinetic; 3 speeds)
         Neuromuscular Economy (Isokinetic; 3 speeds)
      Isometric (Biodex)
         Peak Torque
         Rate of Force Development
         Time to Peak Torque
      Isokinetic (Biodex) (Performed at 3 speeds; slow moderate and fast)
         Peak Torque
         Peak Power
         Rate of Power Development
      Vertical Jump Analysis (Force Plate)
         Peak Force
         Peak Power
         Peak Impulse
         Rate of Power Development
   Circulating Markers
      Myoglobin
      Testosterone
      Glutathione (GSH)
      Ferric Reducing Ability of Plasma (FRAP)
      TBARS (Thiobarbituric Acid Reactive Substances)
      Glutathione Reductase
      Interleukin-8 (IL-8)
      Granulocyte Colony Stimulating Factor (G-CSF)
      Granulocyte-Macrophage Colony Stimulating Factor (GM-CSF)
   Cell Surface Expression on Leukocytes
      Glucocorticoid Receptor
      TNFR1
      CR3
      CD-11b
   Tissue Analysis (Phase I ONLY)
      NF-κβ Signaling Pathway
         TNFR1
         C-Myc
         FADD
         Iκβα
         IKKα/β
         NF-κβ
      Caspase-3
      CD11b
      CD18
      CD68 Macrophages
      CD66b Neutrophils
      ICAM-1
      Interleukin-8 (IL-8)
      Granulocyte Colony Stimulating Factor (G-CSF)
      Granulocyte-Macrophage Colony Stimulating Factor (GM-CSF)

Procedures

Anthropometric Measurements

Anthropometric measurements, including height, body mass, and body fat percentage, were assessed during the BL visit. Body mass (±0.1 kg) and height (±0.1 cm) were measured using a Health-o-meter Professional (Patient Weighing Scale, Model 500 KL, Pelstar, Alsip, Ill., USA). All body composition measures were performed using standardized procedures previously described for collecting skinfold measurement from the chest, abdomen and thigh (Hoffman, 2006) and previously published formulas for calculating body fat percentage (Jackson & Pollock, 1985). All measurements of skinfold were performed by the same researcher using the same pair of skinfold calipers (Caliper-Skinfold-Baseline, Model #MDSP121110, Medline, Mundelein, Ill., USA).

TABLE 2
Performance Measures (Based on sensitivity to muscle damage)

| | | Acute Damaging Protocol Timepoints | | | | | |
|---|---|---|---|---|---|---|---|
| | | (Phase 1 and Phase II) | | | | | |
| | | | D2 | | D3 | D4 | D5 |
| | BL | D1 | Pre | 1 hr | 24 hrs | 48 hrs | 96 hrs |
| Isometric/Isokinetic | ✓ | | ✓ | ✓ | ✓ | ✓ | ✓ |
| Leg Extension/EMG | | | | | | | |
| MVIC | | | | | | | |
| PKT | | | | | | | |
| RFD | | | | | | | |
| Time to Peak | | | | | | | |
| EMG-RMS | | | | | | | |
| EMD | | | | | | | |
| MPF | | | | | | | |
| MEDPF | | | | | | | |
| Isokinetic | | | | | | | |
| PKT | | | | | | | |
| PP | | | | | | | |
| RPD | | | | | | | |
| EMG-RMS | | | | | | | |
| MPF | | | | | | | |
| MEDPF | | | | | | | |
| NME | | | | | | | |
| Vertical Jump on | ✓ | | ✓ | ✓ | ✓ | ✓ | ✓ |
| Force Plate | | | | | | | |
| PKF | | | | | | | |
| PP | | | | | | | |
| PI | | | | | | | |
| RPD | | | | | | | |
| Strength Testing | | ✓ | | | | | |
| Squat | | | | | | | |
| Leg Press | | | | | | | |
| Leg Extension | | | | | | | |

RFD—Rate of force development;
EMD—Electromechanical Delay;
MPF—Mean Power Frequency;
MEDPF—Median Power Frequency;
NME—Neuromuscular Economy;
PKT—Peak Torque;
PKF—Peak Force;
PP—Peak Power;
PI—Peak Impulse;
RPD—Rate of Power Development;
RMS—Root Mean Square;
MVIC—Maximal Voluntary Isometric Contraction

Strength Testing (Phase I and II, D1)

Strength testing was completed on the squat, leg press and leg extension exercises at D1 of the acute damage protocols (phase I and II post-supplementation). Strength was determined for each exercise with 1 repetition maximum (1RM) testing. The 1RM tests were performed using methods previously described (Hoffman, 2006). Prior to beginning strength testing, each participant completed a general and specific warm-up. The general warm up consisted of riding a cycle ergometer for 5 minutes at the subjects' preferred resistance. The specific warm-up consisted of 10 body weight squats, 10 alternating lunges, 10 walking knee hugs and 10 walking butt kicks. Each participant performed two warm-up sets using a resistance level that was approximately 40-60% and 60-80% of his perceived maximum, respectively. The third set was the first attempt at the participant's 1RM. If the set was successfully completed, then weight was added and another set was attempted. If the set was not successfully completed, then the weight was reduced and another set was attempted. A 3-5 min rest period was provided between each set. This process of adding and removing weight was continued until a 1RM has been reached. Attempts not meeting the range of motion criterion for each exercise, as determined by the trainer, were discarded. All 1RM tests were completed under the supervision of a Certified Strength and Conditioning Specialist.

Performance Testing (Phase I and II, D2-D5)

All performance testing were completed at BL and during both AP1 and AP2. During the acute damaging protocols, performance testing occured on D2, D3, D4 and D5. On D2, performance testing took place before the acute damaging protocol (PRE) and 1 hour after the acute damaging protocol (1HR). All performance testing was overseen by a Certified Strength and Conditioning Specialist.

Electromyography (EMG) Methods

To assess muscle activity during performance measures, a bipolar (4.6 cm center-to-center) surface electrode (Quinton Quick-Prep silver-silver chloride) arrangement was placed over the vastus lateralis muscle of the non-biopsied leg, at approximately 66 percent of the line from the anterior superior illiac spine to the superior lateral border of the patella. The reference electrode was placed over the lateral tibial epicondyle. Inter-electrode impedance was kept below 5,000 ohms with shaving and abrasion of the skin beneath the electrodes. The raw EMG signals were pre-amplified using a differential amplifier (MP150 BIOPAC Systems, Inc., Santa Barbara, Calif.), sampled at 1,000 Hz, and stored on a personal computer (Dell Latitude E6530, Dell Inc., Round Rock, Tex.) for off-line analysis.

Using computer software (AcqKnowledge v4.2, BIOPAC Systems, Inc., Santa Barbara, Calif.), we determined the maximal root mean square (RMS) for the vastus lateralis during the maximal voluntary contraction and isokinetic contractions. The RMS value was defined as the amount of muscle activation during contraction. The RMS obtained during the isokinetic contractions was normalized to the RMS value from the MVIC to determine neuromuscular economy (NME) at three different contraction speeds during isokinetic testing. We also calculated mean power frequency (MPF) and median power frequency (MEDPF), measures of conduction velocity, during maximal voluntary isometric contractions and isokinetic contractions. Electromechanical delay (EMD), the amount of time from the increase in EMG activity to the onset of force production, was determined during the maximal voluntary isometric contractions.

Maximal Voluntary Isometric Contraction (MVIC)

Participants were positioned on a BioDex S4 isokinetic dynamometer (Biodex Medical System, Inc., New York, N.Y., USA) in a seated position with the hip at an angle of 110° and strapped to the machine at the waist and shoulders. The research team then lined up the arm of the dynamometer with the participant's non-biopsied leg. The axis of rotation for the dynamometer was lined up with the axis of rotation of the individuals knee. The leg was then be strapped to the arm of the dynamometer just above the lateral malleolus of the ankle. All seat and dynamometer settings were recorded to ensure identical testing conditions. Next, the evaluators positioned the individual's knee at an angle of 110 degrees of extension (180 degrees representing full extension). The arm of the dynamometer was then locked into place so no movement would occur. Participants were given two practice attempts at the MVIC before recording began. The participants were then instructed to exert their maximum strength when trying to extend the knee and to produce the strength as fast as possible. Participants had 2 attempts, each lasting 5 seconds with a 3-minute rest interval between each attempt. This test lasted approximately 10 minutes. From the MVIC, we were able to determine peak torque (PKT) and rate of force development (RFD).

Isokinetic Contractions

After completing the MVIC test, participants then completed a series of isokinetic contractions on the BioDex S4 isokinetic dynamometer. The seat and leg setup were the same as for the MVIC test. Each participant completed 3 sets of isokinetic leg extensions at 30, 120, and 240 degrees per second, respectively. Previous research has utilized isokinetic speeds at 30, 120 and 240 degrees per second to assess muscle function at slow, intermediate and fast speeds (Gur, Gransberg, vanDyke, Knutsson, & Larsson, 2003; Jenkins et al., 2014). The order of the sets were randomized to account for any fatigue from each set. The starting point for a repetition was when the knee was at 90 degrees, with 180 degrees representing full extension. Participants were given two practice attempts at each speed before recording began. Participants were instructed to give maximal effort as they extended the knee joint to 180 degrees. Then they relaxed the muscles to allow the leg to return to the starting position. This process of starting at 90 degrees, extending to 180 degrees and then returning back to 90 degrees was considered 1 repetition. Each set required participants to complete 3 repetitions at the respective speed. Participants were given 3 minutes of rest between each set. This test lasted approximately 10 minutes. From the isokinetic contractions we determined the PKT, RFD, peak power (PP) and rate of power development (RPD). We also calculated the percent difference in power between slow speed contractions and high speed contractions.

Vertical Jump

Participants completed 3 countermovement (vertical) jumps while standing on a force plate (Advance Medical Technology, Inc., Watertown, Mass., USA). Participants stood on the force plate with both feet approximately shoulder width apart. Each participant was instructed to jump straight up as high as they could with both feet leaving the force plate at the same time. Participants then landed on the force plate with both feet. The rest periods between each jump was 3 minutes. This test lasted approximately 10 minutes. From the vertical jump test, we were able to obtain peak force (PKF), PP, peak impulse (PI), and RPD.

Blood Measurements

### Muscle Damage, Inflammation, Oxidative Stress, and Stress Hormones in Blood

Blood samples were obtained from a forearm vein at baseline (BL) using a 20-gauge disposable needle equipped with a Vacutainer® tube holder. During the acute exercise bout post-supplementation in phase I and phase II, blood samples were drawn from one of the subjects' forearm veins using a Teflon™ cannula. During the acute damage protocol, blood draws occured prior to the beginning of the workout, immediately following the workout and one hour after the workout with the cannula. In addition, participants reported to the HPL for a 5-, 24-, 48-, and 96-hour blood sample with the same methods described for baseline (BL) collection. The amount of blood drawn during each of these samples was 30 ml (one draw per timepoint).

TABLE 3
Muscle damage, inflammation, oxidative stress, and stress hormone
measurements.
Muscle damage, inflammation oxidative stress, stress hormones

| | | Blood Draws During Acute | | | | | | |
|---|---|---|---|---|---|---|---|---|
| | | Damaging Protocol Timepoints | | | | | | |
| | | (Phase 1 and Phase II) | | | | | | |
| Time points | BL | Pre | IP | 1 hr | 5 hr | 24 hr | 48 hr | 96 hr |
| CK | | x | | | | x | x | x |
| Testosterone | x | x | x | x | x | x | x | x |
| Cortisol | x | x | x | x | x | x | x | x |
| Glutathione (GSH) | x | x | x | x | x | x | x | x |
| Ferric Reducing | x | x | x | x | x | x | x | x |
| Ability of Plasma | | | | | | | | |
| (FRAP ™)- | | | | | | | | |
| TBARS | x | x | x | x | x | x | x | x |
| (Thiobarbituric Acid | | | | | | | | |
| Reactive Substances) | | | | | | | | |
| Glutathione reductase | x | x | x | x | x | x | x | x |
| Interleukin-8 (IL-8) | x | x | x | x | x | x | x | x |
| Granulocyte Colony | x | x | x | x | x | x | x | x |
| Stimulating Factor | | | | | | | | |
| (G-CSF) | | | | | | | | |
| Granulocyte- | x | x | x | x | x | x | x | x |
| Macrophage Colony | | | | | | | | |
| Stimulating Factor | | | | | | | | |
| (GM-CSF) | | | | | | | | |

Serum and plasma samples were analyzed for each of the following analytes, at the specified timepoints, according to manufacturers' instructions. Each of the assays is discussed below.

Creatine Kinase

Assay kits for creatine kinase were ordered from Sekisui Diagnostics®, Lexington, Mass. (product number: 326-10), who report an intra-assay coefficient of variation of ≦0.4% (U/L≧288) and an inter-assay coefficient of variation of ≦0.4% (U/L≧288), to quantify the creatine kinase concentrations of samples through the use of creatine phosphate and adenosine disphosphate as substrates. The method uses optimized conditions developed jointly by the Scandinavian Committee on Enzymes and the German Society for Clinical Chemistry. The reportable range for this assay kit is 2-1500 U/L.

Cortisol

Assay kit was ordered from Eagle Biosciences, Nashua, N.H. (product number: DK0001). Inter assay variability has been reported <9.8%, while intra assay variability has been reported as <9.0%. The lowest concentration that can be detected is 2.44 ng/mL, while with proper dilustion no maximal detection limit has been established.

Glutathione

Assay kit was ordered from Cayman Chemical Company®, Ann Arbor, Mich. (product number: 703002), who report an intra-assay coefficient of variation of 1.6% (n=83) and an inter-assay coefficient of variation of 3.6% (n=5). This kit quantifies glutathione concentrations, through an enzymatic recycling method, using glutathione reductase. The listed dynamic range of detection is 0-16 μM of glutathione.

Ferric Reducing Ability of Plasma (FRAP)

This assay was ordered from Arbor Assays, Ann Arbor, Mich. (product number: K043-H1). Assay precision has been reported to be less than 5% for both inter and intra-assay variability. Sensitivity of the assay has been determined to be 8.06 μM, while the limit of detection was defined as 5.91 μM.

GSH Reductase

This assay kit was ordered from Cayman Chemical Company®, Ann Arbor, Mich. (product number 703202). Briefly, this assay determined the GSH Reductase activity, by measuring the reduction in absorbance in a kinetic fashion. Inter and intra-assay variation was determined to be less than 10%, while the assay sensitivity was determined to measure any activity greater than 20 nmol/min/ml.

TBARS

Assay kit was ordered from Cayman Chemical Company®, Ann Arbor, Mich. (product number: 10009055), who report an intra-assay coefficient of variation of 5.5% and 7.6% (n=10, and 16, respectively) and an inter-assay coefficient of variation of 5.9% and 5.1% (n=8, and 16, respectively). The assay was used to quantify lipid peroxidation by reacting malondialdehyde with thiobarbituric acid under high temperature and acidic conditions. The reported assay range is between 0 and 50 μM.

Circulating Cytokine Concentrations (IL-8, G-CSF, GM-CSF)

Plasma concentrations of interleukin-8 (IL-8), granulocyte colony stimulating factor (G-CSF) and granulocyte/macrophage colony stimulating factor (GM-CSF) were analyzed via multiplex assay, using the human cytokine/chemokine panel one (EMD Millipore, Billerica, Mass., USA). All samples were thawed once and analyzed in duplicate by the same technician using the MagPix (EMD Millipore), with mean coefficient of variation of 8.04%, 7.82%, and 7.10% for IL-8, G-CSF and GM-CSF respectively.

Immune Cell Modulation in Blood

### Flow Cytometry/Cell Staining

Samples were obtained from fresh, anti-coagulated (K2EDTA), whole blood, and analyzed in duplicate. Erythrocytes were lysed from 350 μl of whole blood with BD Pharminigen Lyse solution (BD Biosciences, Franklin Lakes, N.J.) within 30 min of collection. Samples were then washed in staining buffer containing 1×phosphate-buffered saline containing 0.2% bovine serum albumin (BD Pharminigen Stain Buffer; BD Biosciences) by centrifugation and aspiration three times. Leukocytes were then be re-suspended in 100 μl BD Pharminigen Stain Buffer. Direct staining methods were used to label the target receptors as described herein. Surface staining was completed according to manufacturers' instructions. Cells were then be re-suspended in 1.0 ml of stain buffer for flow cytometry analysis.

Analysis

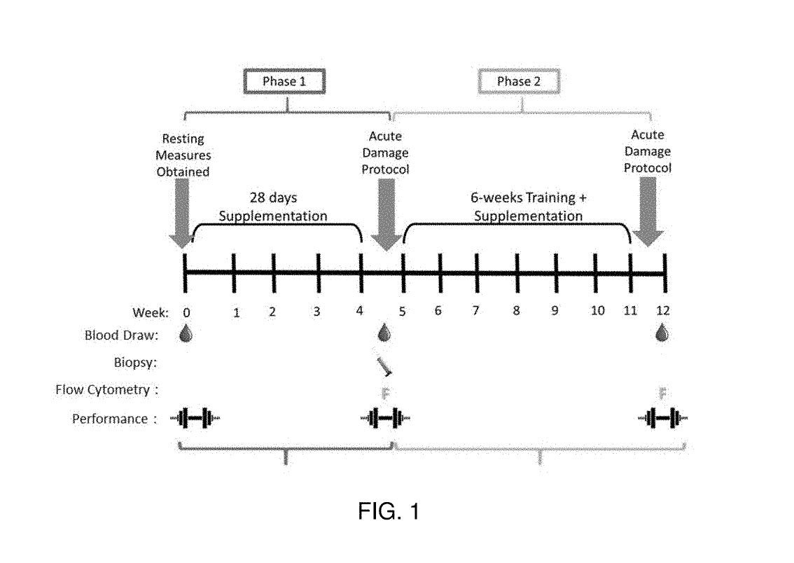

Flow cytometry analysis of stained cells were run on a BD C6 Accuri Flow Cytometer (BD Biosciences, San Jose, Calif.); equipped with BD Accuri analysis software (BD Biosciences). Forward and side scatter along with four fluorescent channels of data were collected using two lasers providing excitation at 488 and 640 nm. Granulocytes, lymphocytes and monocytes were determined by initial gating based on forward and side scatter. Monocytes were also determined by gating for CD14+ cells as also described previously (Tallone et al., 2011). A minimum of 10,000 events, defined as CD14+ monocytes, will be obtained with each sample (FIG. 1).

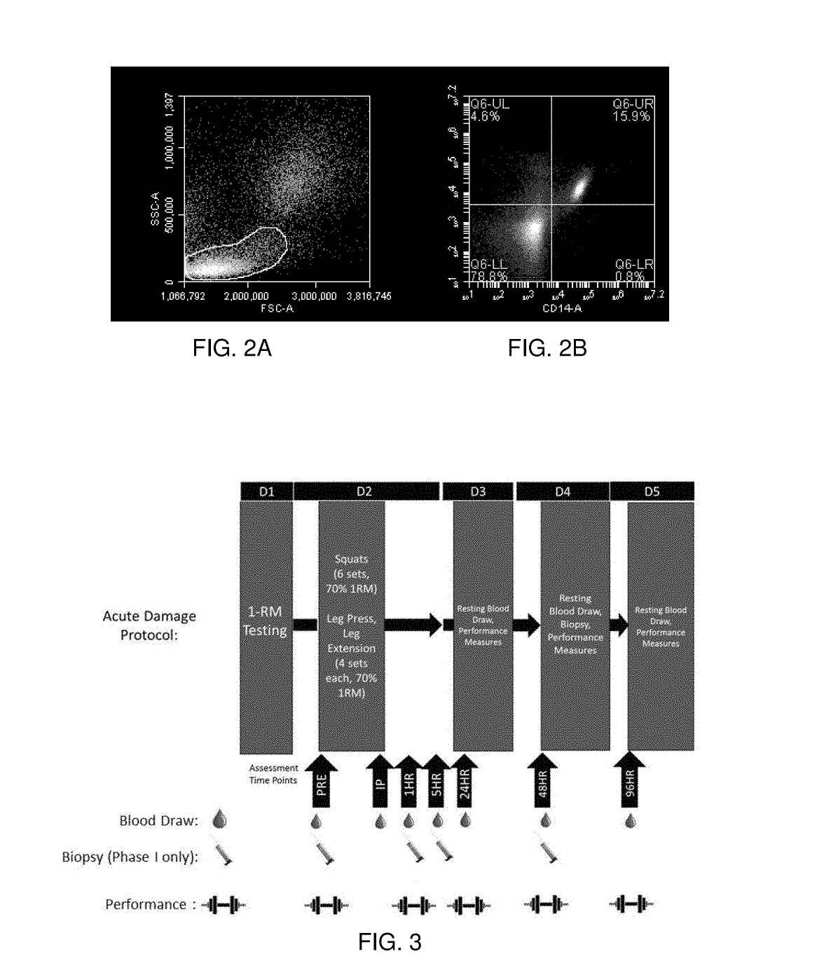

Analysis of monocyte subpopulations was completed by quadrant analyses, in which CD14 was compared with the markers of interest. Mean fluorescence of the markers of interest on CD14+ cells was recorded, representing the expression per cell (Fragala et al., 2011). Proportion of positive versus negative expression was determined by quadrant analysis (FIG. 2). Compensation for fluorescence spillover was set based on manufacturer recommendations (BD Biosciences).

TABLE 4
Flow Cytometry results.
Flow Cytometry

| | | | Acute Damaging Protocol Timepoints | | | | | | |
|---|---|---|---|---|---|---|---|---|---|
| | | | (Phase 1 and Phase II) | | | | | | |
| Time Points | Description of Marker | BL | Pre | IP | 1 hr | 5 hr | 24 hr | 48 hr | 96 hr |
| TNFR1 | Tags TNF-α receptor 1 | | x | x | x | x | x | X | |
| | complexes. Identified on | | | | | | | | |
| | monocytes and granulocytes. | | | | | | | | |
| GCR | Tags glucocorticoid | | x | x | x | x | x | X | |
| | receptors. Identified on | | | | | | | | |
| | lymphocytes; current work in | | | | | | | | |
| | our lab has identified this on | | | | | | | | |
| | granulocytes; monocytes | | | | | | | | |
| | may express at later | | | | | | | | |
| | timepoints | | | | | | | | |
| CD14 | Identifies monocytes | | x | x | x | x | x | X | |
| CD4 | Identifies T-lymphocytes | | x | x | x | x | x | X | |
| CD3 | Identifies T-lymphocytes | | x | x | x | x | x | X | |
| CD56 | Identifies NK cells | | x | x | x | x | x | X | |
| CR3 | Identifies the CD11b/CD18 | | x | x | x | x | x | X | |
| | integrin molecule, signaling | | | | | | | | |
| | cell adhesion to the vascular | | | | | | | | |
| | surface. Identified on | | | | | | | | |
| | monocytes and granulocytes | | | | | | | | |

Tissue Measurements

### Biopsy Procedures

The fine needle muscle biopsy involves the removal of a small piece of muscle tissue from one of the leg muscles using a sterile hollow needle. Fine needle biopsies are a new minimally invasive technique which allows a researcher to obtain multiple muscle samples without large incisions that accompany other methods such as the Bergstrom technique. Additionally, the pain level of a fine needle biopsy has been reported to be minimal with individuals comparing it to a “pushing sensation” with most participants engaging in normal physical activity the same day (Paoli, Pacelli, Toniolo, Miotti, & Reggiani, 2010). Prior to the biopsy, an ultrasound machine was used on the biopsy leg and the images were used to determine proper depth of the biopsy needle. The participant's self-reported dominant leg was used for all biopsies throughout phase I. The area over the outside of the lower thigh muscle (vastus lateralis muscle) was carefully cleaned. A small amount of lidocaine (anesthetic) was injected into and under the skin. Some participants reported a small pinching sensation while the numbing agent is injected. After the area was completely numbed, a small incision to the skin was made and the insertion cannula was placed perpendicular to the muscle until the fascia is pierced. Following placement of the biopsy needle into the biopsy device, the unit was inserted through the cannula. A muscle sample was obtained by the activation of a trigger button, which unloads the spring and activates the needle to collect a small muscle sample. The biopsy needle was then slid out of the insertion cannula while the cannula is maintained in place, thus avoiding repeated skin punctures. During the time that the sample was taken (about 5 seconds) participants may have felt the sensation of pressure in the thigh and on some occasions, this is moderately painful. However, any reported discomfort very quickly passed and the participants were quite capable of performing exercise and daily activities. There may be some minimal bleeding when the needle was removed which may require application of pressure for a few minutes. Following the biopsy, the incision was treated with a sterile dressing and wrapped in a bandage.

The whole biopsy procedure was repeated up to 6 times per timepoint in order to obtain sufficient muscle tissue. The sum of the weights of the biopsy specimens obtained was recorded. All muscle biopsies were performed by a licensed physician or a physician approved technician. All tissue samples were immediately frozen in liquid nitrogen and stored in a −80° C. freezer for subsequent analysis.

Tissue Signaling Analysis

Tissue samples collected during the muscle biopsy procedure were removed from −80° C. and transferred to a conical tube on ice for preparation and homogenization. Each sample was washed with phosphate-buffered saline (PBS), centrifuged for 1 minute at 5,000 RPM, and excess PBS was subsequently aspirated from the conical tube. A lysis buffer with protease inhibitor (EMD Millipore, Billerica, Mass., USA) was then be added to each sample at a rate of 500 μl per 10 mg of tissue. Samples were then be homogenized using a Teflon pestle and sonication (Branson, Danbury, Conn., USA). Tissue samples were placed on a plate shaker (Thermo Fisher Scientific Inc., Waltham, Mass., USA) for 10 minutes at 4° C. and subsequently centrifuged at 10,000 RPM for 5 minutes. The supernatant was then aspirated and used for analysis.

Multiplex enzyme-linked immunosorbent assays (ELISA) were used to quantify total proteins (Caspase 3, 8, 9) and the phosphorylation status of proteins specific to the NF-κβ intracellular signaling pathway using MAGPIX® (Luminex, Austin, Tex., USA) and a multiplex NF-κβ signaling assay kit (EMD Millipore, Billerica, Mass., USA) according to manufacturer's guidelines. Total protein quantification was conducted using a DC protein assay kit (Bio-Rad, Hercules, Calif., USA). Phospho-protein values were then normalized for total protein added per well and are therefore reported as arbitrary units (AU). Other markers of oxidative stress were analyzed by ELISA to determine the oxidative stress related to the resistance training protocol (as described previously). To eliminate inter-assay variance, all tissue samples were thawed once and analyzed in duplicate in the same assay run by a single technician.

TABLE 5
Muscle Tissue Biopsy (Phase I only)

| | | Acute Damaging Protocol (Phase I only) | | | | | | |
|---|---|---|---|---|---|---|---|---|
| Time Points | BL | Pre | IP | 1 hr | 5 hr | 24 hr | 48 hr | 96 hr |
| NF-κβ Signaling | | X | | x | | | x | |
| pathway | | | | | | | | |
| TNFR1 | | | | | | | | |
| C-Myc | | | | | | | | |
| FADD (Ser194) | | | | | | | | |
| IκBα (Ser32) | | | | | | | | |
| IKKα/β (Ser177/ | | | | | | | | |
| Ser181) | | | | | | | | |
| NFκB (Ser536) | | | | | | | | |
| Apoptotic signaling | | X | | x | | | x | |
| (Caspase 3, 8, 9) | | | | | | | | |
| Markers of | | | | | | | | |
| phosphorylation status | | | | | | | | |
| (JNK, FADD, | | | | | | | | |
| p53, BAD, Bcl-2). | | | | | | | | |
| CD11b | | X | | x | | | x | |
| CD18 | | X | | x | | | x | |
| Interleukin-8 (IL-8) | | X | | x | | | x | |
| Granulocyte Colony | | X | | x | | | x | |
| Stimulating Factor | | | | | | | | |
| (G-CSF) | | | | | | | | |
| Granulocyte- | | X | | x | | | x | |
| MacrophageColony | | | | | | | | |
| Stimulating Factor | | | | | | | | |
| (GM-CSF) | | | | | | | | |
| CD68 Macrophages | | x | | x | | | x | |
| CD66b Neutrophils | | x | | x | | | x | |
| ICAM-1 | | x | | x | | | x | |

Study Product and Dosing Regimen

The study products were packaged in sealed bottles containing 200 capsules consumed at 2 capsules b.i.d. (total 4 capsules/day; 2 g/day) with food. Capsules (prepared by Five-Star Pharmacy, Clive, Iowa) are size 00 and white-opaque in color containing:

TABLE 6
Product description.

| XSurge Dosage | | |
|---|---|---|
| (per capsule) | XSurge (%)¹ | Excipient (%)² |
| 500 mg | 97 | 3 |
| 0 g (placebo) | 0 | 100 |

¹XSurge is a 100% water-extracted, granulated, free-flowing, dry powder polyphenolic blend from black tea (Camellia sinensis) and green tea (Camellia sinensis) containing at least 40% total polyphenols and 1.3% theaflavins.
²Avicel PH 105 (microcrystalline cellulose).

Statistical Analysis

Analysis (CON and Experimental Populations) consisted of group×time interactions for all variables measured using repeated measure ANOVA or ANCOVA. Phase I (Untrained) and Phase II (Trained) were treated as separate data sets, and were treated accordingly. Therefore, during Phase I, the CON group, PL group and XS group were compared, while during phase II the PL group and XS group were compared.

Results:

Descriptive Data

Phase 1:

Effects of 28 Days of Supplementation (Comparison of Baseline to Pre-exercise Value in AP1-referred to Here at Post Supplementation)

Performance Measures

### Isokinetic Leg Extension EMG-RMS Measures

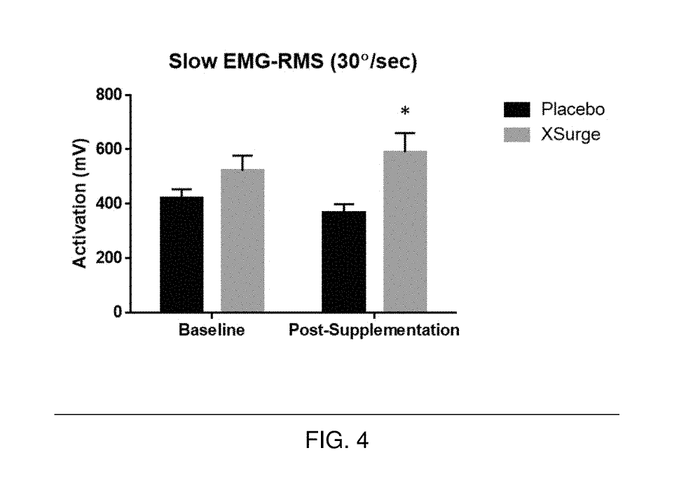

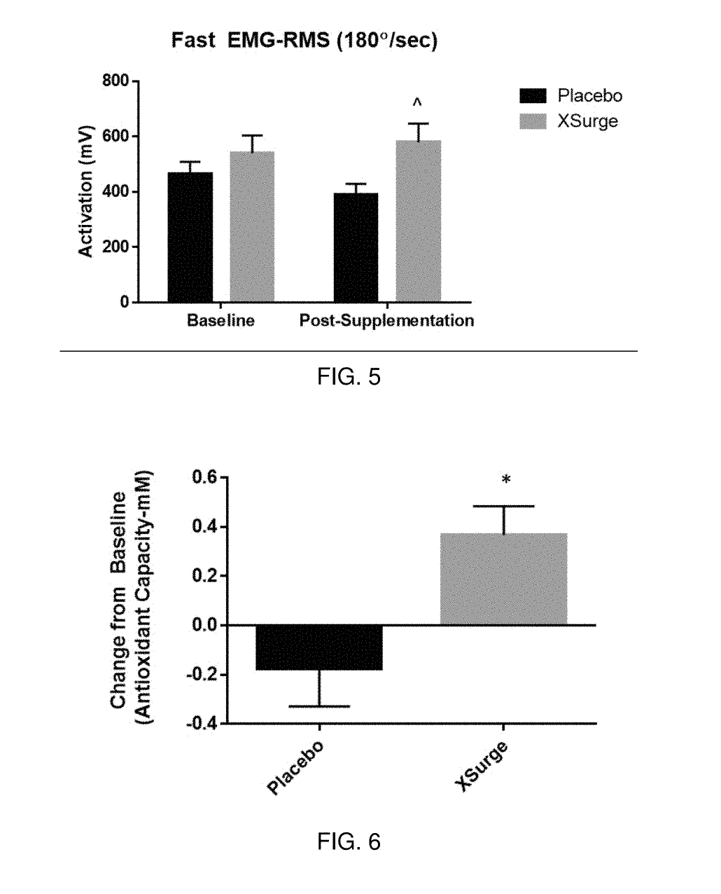

Differences were identified with isokinetic leg extension EMG-RMS measurements at both slow (30° /sec, FIG. 4) and fast (180° /sec, FIG. 5). XS group had significantly higher EMG-RMS muscle signaling (indicative of muscle activation) following 28 days of supplementation than those individuals in the PL group.

Blood Measurements

### Muscle Damage, Inflammation, Oxidative Stress, and Stress Hormones in Blood

Antioxidant Capacity

Four weeks of supplementation with XS increased serum antioxidant capacity as measured by antioxidant capacity assay in comparison to PL (FIG. 6). The change reflected is from the baseline value to the pre-exercise value in AP1, immediately prior to acute damaging muscle protocol and muscle biopsies. The current study confirms previous data on this ingredient which showed the ability of 13 weeks of supplementation to improve antioxidant capacity of the blood.

Cortisol

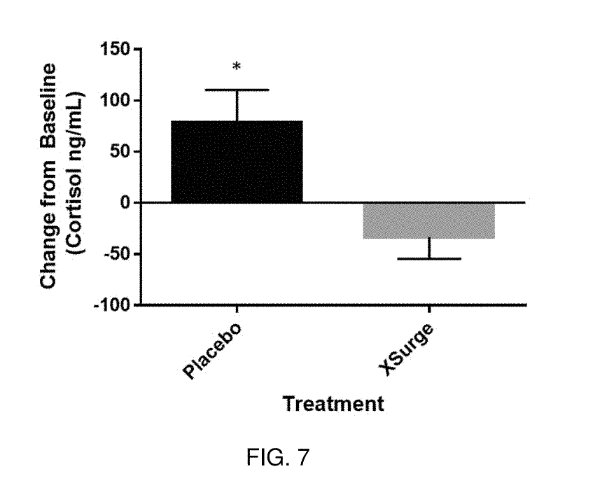

Four weeks of supplementation with XS normalized and prevented the increases in cortisol levels in comparison to the increased cortisol observed in the PL group (FIG. 7). The change reflected is from the baseline value to the pre-exercise value in AP1, immediately prior to acute damaging muscle protocol and muscle biopsies.

GSH Reductase

GSH reductase increased in the XS group following four weeks of supplementation in comparison to the PL group. An increase of 47% was observed in the XS group from the baseline value to the pre-exercise value at AP1, immediately prior to acute damaging muscle protocol and muscle biopsies, in comparison to the 8% increase in the PL group (P=0.042).

Phase 1:

### Effects of 28 Days of Supplementation on Recovery From an Acute Damaging Protocol.

### Performance Measures

### Isokinetic Leg Extension PKT

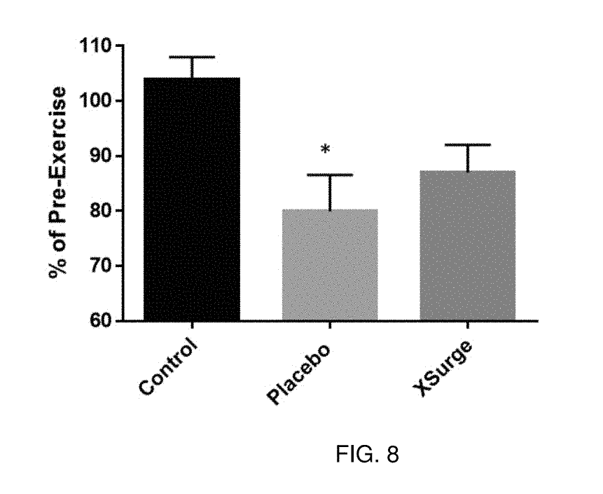

Loss of strength from pre-exercise values at identified at 96 hours following an acute damaging protocol which was conducted following supplementation (FIG. 8). Loss of strength as measured by peak torque (isokinetically at moderate speed from Biodex) was not different in individuals who consumed XS compared to CON subjects (non-exercise group) who did not undergo the acute muscle damaging protocol. However, individuals who consumed PL had significantly increased loss of strength compared to CON group. These data confirm previous results showing XS improved recovery as measured by peak torque following 13 weeks of supplementation.

Vertical Jump on Force Plate PP

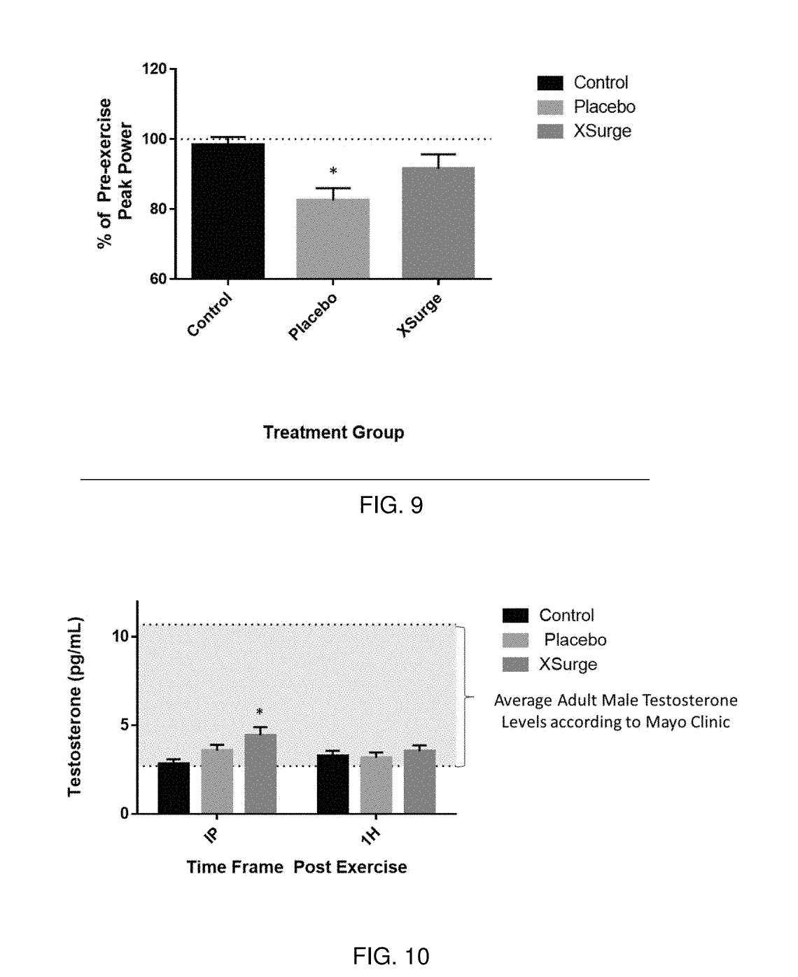

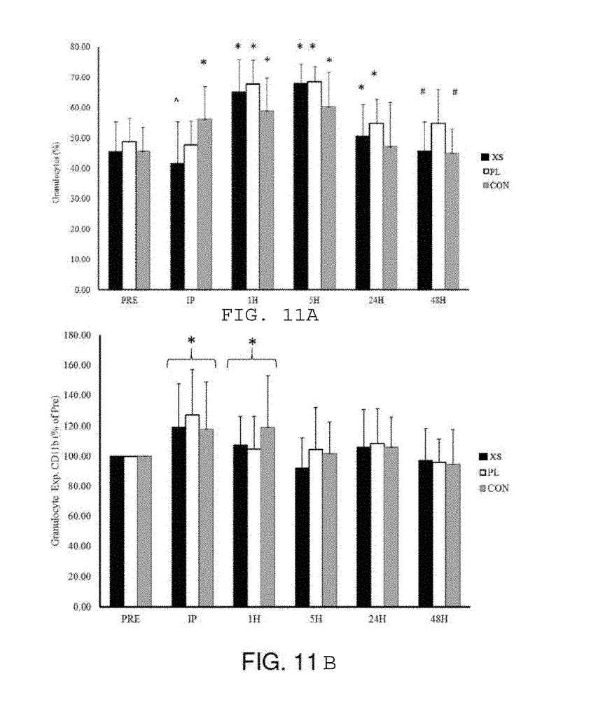

Loss of peak power from pre-exercise values were identified 1 hour following an acute damaging protocol which was conducted following supplementation (FIG. 9). Loss of peak power in verticle jump was not different in individuals who consumed XS compared to CON subjects (non-exercise group) who did not undergo the acute muscle damaging protocol. However, individuals who consumed PL had significantly less peak power compared to CON group.

Blood Measurements

### Muscle Damage, Inflammation, Oxidative Stress, Cellular Changes, and Stress Hormones in Blood

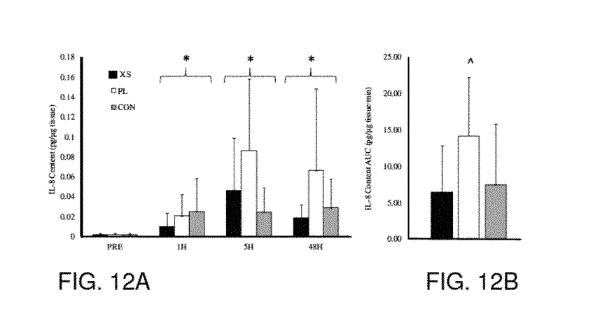

Changes in the granulocyte percentage (CD11b, FIG. 12) in circulation in response to resistance exercise. Granulocyte percentage for PL was significantly greater at 48H than CON (p=0.026) or XS (p=0.022).

Testosterone

Individuals consuming XS had an increased testosterone concentration immediately post exercise in comparison to the CON group which had not completed the muscle damaging protocol (FIG. 10). Although all values were still within the normal values for adult males. Any differences identified were not longer present at 1 hour post exercise.

Intramuscular Protein Content

Magnitude based inferences indicated a “likely” decrease in total Caspase 3 and “possibly” decreased total Caspase 9 in XS compared to PL from PRE-5H. JNK phosphorylation was “likely” decreased from PRE-5H in XS compared to PL. BAD was “very likely” decreased from PRE-5H in XS when compared to PL and Bcl-2 was “likely” decreased from PRE-1H and PRE-5H in XS compared to PL. Phosphorylation of p53 was “likely increased” in XS compared to PL from PRE-1H and PRE-48H.

Intramuscular IL-8 protein content is depicted in FIG. 12. When groups were collapsed, pairwise comparisons indicated that skeletal muscle content of IL-8 was significantly increased compared to PRE at 1H (p<0.001), 5H (p<0.001) and 48H (p<0.001). Additionally, when collapsed across time, pairwise comparisons indicated PL was significantly greater than CON (p=0.024) and XS (p=0.010). Furthermore, AUC analysis indicated a significant difference between groups (F=4.090; p=0.030). Post-hoc analysis indicated PL was significantly greater than XS (p=0.011), while a trend toward a difference was observed between PL and CON (p=0.066).

### SUMMARY

### Phase 1:

### Effects of 28 Days of Supplementation (Comparison of Baseline to Pre-exercise Value in AP1)

Performance Measures

### Isokinetic Leg Extension EMG-RMS Measures

Increased EMG-RMS at both the slow and fast speeds indicate improved muscular activation following consumption of XS for 28 days.

Blood Measurements

### Muscle Damage, Inflammation, Oxidative Stress, and Stress Hormones in Blood

XS supplementation resulted in increased antioxidant capacity and GSH reductase while preventing the increased cortisol response. Oxidative stress is a biological phenomenon marked by an imbalance between reactive free radical production and the antioxidant defense system. Acute exercise increases free radical production, intensifies oxidative stress, causes cellular damage, and can increase stress in the body. Since GSH reductase catalyzes the reduction of glutathione disulfide (GSSG) to glutathione (GSH), a molecule with a crucial role in resisting oxidative stress and maintaining the reducing environment of the cell, the ability to increase GSH reductase would assist in preventing oxidative stress and promoting antioxidant/oxidant balance of the cell.

Because exercise can induce the production of free radicals that when present in excess can damage tissue, the ability to improve the antioxidant capacity (shown in the data above) of the blood in individuals with XS supplementation is indicative of increased ability to protect the body and/or tissues. In addition, XS supplementation prevented the increases in cortisol stress response that occurred in the current study and increased GSH reductase thus promoting the cellular protection against oxidative stress.

Phase 1:

### Effects of 28 Days of Supplementation on Recovery From an Acute Damaging Protocol.

Performance Measures

### Isokinetic Leg Extension PKT and Vertical Jump on Force Plate PP

These data show that supplementation for 28 days with XS resulted in improved recovery from an acute muscle damaging protocol. Recovery of strength at measured by peak torque occurred by 96 hours post exercise (further supporting previous data for this ingredient). Furthermore, in addition to the benefits at 96 hours, evaluation of vertical jump showed more immediate recovery benefits of XS supplementation were apparent at 1 hour post acute damaging protocol.

Blood Measurements

### Muscle Damage, Inflammation, Oxidative Stress, Cellular Changes, and Stress Hormones in Blood

Response of testosterone to exercise has been reported to be variable in the literature. While some individuals may see a slight increase in testosterone immediately after exercise, elite athletes can see a decrease in testosterone. This decrease in testerone can be a sign that they are harming their body as an increased testosterone has been reported to be associated with improved energy, muscle growth and strength. Supplementation with XS improved testosterone levels immediately post exercise.

Since granulocyte percentage for placebo was higher than with XS and given the chemoattractant properties of IL-8 for neutrophils, the reduced granulocyte proportion observed in this investigation may be related to the reduced intramuscular IL-8.

Tissue Measurements XS attenuated indices of apoptosis in skeletal muscle following an acute muscle-damaging resistance exercise.

IL-8 serves as a chemoattractant and increases following resistance exercise. In agreement with the literature, our results indicated a significant elevation in intramuscular IL-8 protein content following resistance exercise. Furthermore, AUC analysis revealed a reduced exercise response following XS supplementation. Therefore, our results indicate XS supplementation may reduce the IL-8 response to resistance exercise. Therefore, the reduced IL-8 response in skeletal muscle following resistance exercise may represent a reduced pro-inflammatory response that is promoted by XS supplementation following acute resistance exercise. In response to XS supplementation, we observed a significantly greater circulating granulocyte percentage in PL than XS and CON at 48 H. These findings may implicate XS supplementation as a means to reduce the pro-inflammatory response following resistance exercise.

The foregoing description and drawings comprise illustrative embodiments of the present invention. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art who have the disclosure before them will be able to make modifications and variations therein without departing from the scope of the invention.

### REFERENCES

Clarkson, P. M., & Hubal, M. J. (2002). Exercise-induced muscle damage in humans. Am J Phys Med Rehabil, 81(11 Suppl), S52-69. doi: 10.1097/01.PHM.0000029772.45258.43
Della Gatta, P. A., Cameron-Smith, D., & Peake, J. M. (2014). Acute resistance exercise increases the expression of chemotactic factors within skeletal muscle. Eur J Appl Physiol, 114(10), 2157-2167. doi: 10.1007/s00421-014-2936-4
Fischer, C. P., Hiscock, N. J., Penkowa, M., Basu, S., Vessby, B., Kallner, A., . . . Pedersen, B. K. (2004). Supplementation with vitamins C and E inhibits the release of interleukin-6 from contracting human skeletal muscle. J Physiol, 558(Pt 2), 633-645. doi: 10.1113/jphysio1.2004.066779
Fragala, M. S., Kraemer, W. J., Mastro, A. M., Denegar, C. R., Volek, J. S., Hakkinen, K., . . . Maresh, C. M. (2011). Leukocyte beta2-adrenergic receptor expression in response to resistance exercise. Med Sci Sports Exerc, 43(8), 1422-1432. doi: 10.1249/MSS.0b013e31820b88bc
Francisco-Cruz, A., Aguilar-Santelises, M., Ramos-Espinosa, O., Mata-Espinosa, D., Marquina-Castillo, B., Barrios-Payan, J., & Hernandez-Pando, R. (2014). Granulocyte-macrophage colony-stimulating factor: not just another haematopoietic growth factor. Med Oncol, 31(1), 774. doi: 10.1007/s12032-013-0774-6
Freidenreich, D. J., & Volek, J. S. (2012). Immune responses to resistance exercise. Exerc Immunol Rev, 18, 8-41.
Gur, H., Gransberg, L., vanDyke, D., Knutsson, E., & Larsson, L. (2003). Relationship between in vivo muscle force at different speeds of isokinetic movements and myosin isoform expression in men and women. Eur J Appl Physiol, 88(6), 487-496. doi: 10.1007/s00421-002-0760-8
Hammond, M. E., Lapointe, G. R., Feucht, P. H., Hilt, S., Gallegos, C. A., Gordon, C. A., . . . Tekamp-Olson, P. (1995). IL-8 induces neutrophil chemotaxis predominantly via type I IL-8 receptors. J. Immunol, 155(3), 1428-1433.
Hoffman, J. R. (2006). Norms for fitness, performance, and health. Champaign: Human Kinetics.
Hoffman, J. R., Im, J., Kang, J., Maresh, C. M., Kraemer, W. J., French, D., . . . Chance, B. (2007). Comparison of low- and high-intensity resistance exercise on lipid peroxidation: role of muscle oxygenation. J Strength Cond Res, 21(1), 118-122. doi: 10.1519/R-20526.1
Hudson, M. B., Hosick, P. A., McCaulley, G. O., Schrieber, L., Wrieden, J., McAnulty, S. R., . . . Quindry, J. C. (2008). The effect of resistance exercise on humoral markers of oxidative stress. Med Sci Sports Exerc, 40(3), 542-548. doi: 10.1249/MSS.0b013e31815daf89
Jackson, A. S., & Pollock, M. L. (1985). Practical Assessment of Body-Composition. Physician and Sportsmedicine, 13(5), 76.
Jenkins, N. D., Housh, T. J., Cochrane, K. C., Bergstrom, H. C., Traylor, D. A., Lewis, R. W., Jr., . . . Cramer, J. T. (2014). Effects of anatabine and unilateral maximal eccentric isokinetic muscle actions on serum markers of muscle damage and inflammation. Eur J Pharmacol, 728, 161-166. doi: 10.1016/j.ejphar.2014.01.054
Nguyen, H. X., & Tidball, J. G. (2003). Null mutation of gp91phox reduces muscle membrane lysis during muscle inflammation in mice. J Physiol, 553(Pt 3), 833-841. doi: 10.1113/jphysiol.2003.051912
Nieman, D. C., Davis, J. M., Brown, V. A., Henson, D. A., Dumke, C. L., Utter, A. C., . . . McAnulty, L. S. (2004). Influence of carbohydrate ingestion on immune changes after 2 h of intensive resistance training. J Appl Physiol (1985), 96(4), 1292-1298. doi: 10.1152/japplphysiol.01064.2003
Nieman, D. C., Henson, D. A., McAnulty, S. R., McAnulty, L., Swick, N. S., Utter, A. C., . . . Morrow, J. D. (2002). Influence of vitamin C supplementation on oxidative and immune changes after an ultramarathon. J Appl Physiol (1985), 92(5), 1970-1977. doi: 10.1152/japplphysiol.00961.2001
Paoli, A., Pacelli, Q. F., Toniolo, L., Miotti, D., & Reggiani, C. (2010). Latissimus dorsi fine needle muscle biopsy: a novel and efficient approach to study proximal muscles of upper limbs. J Surg Res, 164(2), e257-263. doi: 10.1016/j.jss.2010.05.043
Parkin, J., & Cohen, B. (2001). An overview of the immune system. Lancet, 357(9270), 1777-1789. doi: 10.1016/S0140-6736(00)04904-7
Paulsen, G., Benestad, H. B., Strom-Gundersen, I., Morkrid, L., Lappegard, K. T., & Raastad, T. (2005). Delayed leukocytosis and cytokine response to high-force eccentric exercise. Med Sci Sports Exerc, 37(11), 1877-1883.
Paulsen, G., Crameri, R., Benestad, H. B., Fjeld, J. G., Morkrid, L., Hallen, J., & Raastad, T. (2010). Time course of leukocyte accumulation in human muscle after eccentric exercise. Med Sci Sports Exerc, 42(1), 75-85. doi: 10.1249/MSS.0b013e3181ac7adb
Petersen, E. W., Ostrowski, K., Ibfelt, T., Richelle, M., Offord, E., Halkjaer-Kristensen, J., & Pedersen, B. K. (2001). Effect of vitamin supplementation on cytokine response and on muscle damage after strenuous exercise. Am J Physiol Cell Physiol, 280(6), C1570-1575.
Pizza, F. X., Peterson, J. M., Baas, J. H., & Koh, T. J. (2005). Neutrophils contribute to muscle injury and impair its resolution after lengthening contractions in mice. J Physiol, 562(Pt 3), 899-913. doi: 10.1113/jphysiol.2004.073965
Ramel, A., Wagner, K. H., & Elmadfa, I. (2004). Plasma antioxidants and lipid oxidation after submaximal resistance exercise in men. Eur J Nutr., 43(1), 2-6. doi: 10.1007/s00394-004-0432-z
Reid, M. B., & Durham, W. J. (2002). Generation of reactive oxygen and nitrogen species in contracting skeletal muscle: potential impact on aging. Ann N Y Acad Sci, 959, 108-116.
Roberts, A. W. (2005). G-CSF: a key regulator of neutrophil production, but that's not all! Growth Factors, 23(1), 33-41. doi: 10.1080/08977190500055836
Tallone, T., Turconi, G., Soldati, G., Pedrazzini, G., Moccetti, T., & Vassalli, G. (2011). Heterogeneity of human monocytes: an optimized four-color flow cytometry protocol for analysis of monocyte subsets. J Cardiovasc Transl Res, 4(2), 211-219. doi: 10.1007/s12265-011-9256-4
Tidball, J. G., & Villalta, S. A. (2010). Regulatory interactions between muscle and the immune system during muscle regeneration. Am J Physiol Regul Integr Comp Physiol, 298(5), R1173-1187. doi: 10.115²/ₐjpregu.00735.2009
Vassilakopoulos, T., Karatza, M. H., Katsaounou, P., Kollintza, A., Zakynthinos, S., & Roussos, C. (2003). Antioxidants attenuate the plasma cytokine response to exercise in humans. J Appl Physiol (1985), 94(3), 1025-1032. doi: 10.1152/japplphysio1.00735.2002
Vassilakopoulos, T., Roussos, C., & Zakynthinos, S. (2005). When are antioxidants effective in blunting the cytokine response to exercise? Med Sci Sports Exerc, 37(2), 342-343; author reply 344.
Watson, T. A., Callister, R., Taylor, R. D., Sibbritt, D. W., MacDonald-Wicks, L. K., & Garg, M. L. (2005). Antioxidant restriction and oxidative stress in short-duration exhaustive exercise. Med Sci Sports Exerc, 37(1), 63-71.

We claim:

1. A method of improving muscle performance comprising administering to a subject an efficacious dose of a mixture of water soluble extracts of green and black tea.

2. The method of claim 1 wherein muscle performance includes muscle signaling, neuromuscular activation, or neuromuscular performance.

3. The method of claim 1 wherein muscle performance includes muscle response or reaction time.

4. A method of improving cellular protection from oxidative damage by increasing antioxidant capacity and/or glutathione reductase in a subject comprising administering to a subject an efficacious dose of a mixture of water soluble extracts of green and black tea.

5. A method of improving or sustaining cortisol stress response in the subject following exercise comprising administering to the subject an efficacious dose of a mixture of water soluble extracts of green and black tea.

6. A method of improving testosterone levels in a subject following exercise comprising administering to the subject an efficacious dose of a mixture of water soluble extracts of green and black tea.

7. The method of claim 1, further comprising administering the mixture following a muscle damaging exercise regimen in order to reduce muscle inflammation.

8. The method of claim 1, to reduce the pro-inflammatory cytokine response and subsequent cellular damage following resistance exercise.

9. A method for supporting a healthy immune response during a resistance exercise program comprising administering to a subject an efficacious dose of a mixture of water soluble extracts of green and black tea.
